(19) United States
(12) Patent Application Publication
(10) Pub. No.: US 2025/0099081 A1
(43) Pub. Date: Mar. 27, 2025

(54) THREE-DIMENSIONAL ULTRASOUND IMAGE PROCESSING APPARATUS

(71) Applicant: FUJIFILM Corporation, Tokyo (JP)

(72) Inventor: Motochika Shimizu, Tokyo (JP)

(21) Appl. No.: 18/884,782

(22) Filed: Sep. 13, 2024

(30) Foreign Application Priority Data
Sep. 25, 2023 (JP) 2023-160130

(51) Int. Cl. A61B 8/08; A61B 8/06; G01S 7/52; G06T 5/20; G06T 5/50; G06T 5/70; G06T 7/00; G06T 7/593

(52) U.S. Cl. CPC A61B 8/5269; A61B 8/483; G01S 7/52077; G06T 5/20; G06T 5/50; G06T 5/70; G06T 7/0012; G06T 7/593; A61B 8/06; A61B 8/488; G06T 2200/04; G06T 2207/10136; G06T 2207/30104

(57) ABSTRACT

An information processing unit acquires spatial frequency distribution data in a depth direction for one frame in two-dimensional ultrasound image data of a plurality of frames constituting three-dimensional ultrasound image data, and performs filter processing, based on filter characteristics determined in accordance with the spatial frequency distribution data and characteristics of transmitted ultrasound in which each two-dimensional ultrasound image data is acquired. The information processing unit searches for a spatial frequency corresponding to a maximal value of a distribution indicated by the spatial frequency distribution data in a search range determined in accordance with a pulse width of the transmitted ultrasound, and obtains characteristics for suppressing a level of the spatial frequency distribution in a spatial frequency band including the spatial frequency that is searched for.

### CROSS REFERENCE TO RELATED APPLICATION

The present application claims priority from Japanese Patent Application No. 2023-160130, filed Sep. 25, 2023, the content of which is hereby incorporated by reference into this application.

### BACKGROUND OF THE INVENTION

### 1. Field of the Invention

The present disclosure relates to a three-dimensional ultrasound image processing apparatus, and particularly to an apparatus that performs filter processing on two-dimensional ultrasound image data constituting three-dimensional ultrasound image data.

### 2. Description of the Related Art

There is an ultrasound diagnostic apparatus that acquires B-mode image data of a plurality of frames at different positions in a subject to acquire three-dimensional ultrasound image data. Such an ultrasound diagnostic apparatus has a two-dimensional array probe as an ultrasound probe, and repeatedly acquires the B-mode image data while performing electronic scanning not only in a major axis direction but also in a minor axis direction (depth direction). Each B-mode image data is acquired, for example, by scanning with a transmission beam on a plane. The B-mode image data of one frame is generated by receiving the reflected ultrasound arriving from each direction in which the transmission beam scanned on one plane is directed. The B-mode images of the plurality of frames connected in a minor axis scanning direction constitute a three-dimensional ultrasound image.

In the ultrasound diagnostic apparatus, the ultrasound that is transmitted earlier is reciprocated, and then the next ultrasound is transmitted. Therefore, there is an upper limit on the number of times of transmission and reception per unit time, and there is also an upper limit on a frame rate (also referred to as a volume rate in three-dimensional imaging). In order to increase the frame rate and reduce a time for acquiring the three-dimensional ultrasound image data, an interval in a case of scanning with the transmission beam is increased as compared with a case of acquiring the B-mode image data on one plane. The number of transmission beams per frame in a case of acquiring the three-dimensional ultrasound image data is decreased as compared with a case of generating only the B-mode image data on one plane. Further, a spatial resolution of the B-mode image is improved by forming a plurality of reception beams for one transmission beam and generating the B-mode image data based on a plurality of reception signals obtained from the plurality of reception beams, that is, signals obtained by parallel reception phasing.

It should be noted that JP2016-87302A discloses parallel reception phasing. JP2020-69304A discloses that, as a technology related to the present disclosure, filter processing is performed on a spatial frequency distribution for two axes of a depth direction and an azimuthal angle direction of an ultrasound image.

### SUMMARY OF THE INVENTION

In the ultrasound image such as the B-mode image and the Doppler image generated by the parallel reception phasing, due to the pixel value undulating in the depth direction in which the ultrasound is transmitted and received, as well as in a scanning direction of the transmission beam (azimuthal angle direction in sector scanning), spot-like speckles extending in the scanning direction may be generated. As a result, a stripe pattern extending in the scanning direction may appear as an artifact. The stripe pattern tends to be more remarkable as the scanning interval of the transmission beam is larger.

An object of the present disclosure is to suppress an artifact caused by a pixel value undulating in a depth direction of the ultrasound image for a plurality of ultrasound images constituting a three-dimensional ultrasound image.

An aspect of the present disclosure relates to a three-dimensional ultrasound image processing apparatus comprising: an information processing unit that executes processing of acquiring spatial frequency distribution data in a depth direction for one frame in two-dimensional ultrasound image data of a plurality of frames constituting three-dimensional ultrasound image data, and processing of performing filter processing, which is based on filter characteristics determined in accordance with the spatial frequency distribution data and characteristics of transmitted ultrasound in a case in which each two-dimensional ultrasound image data is acquired, on each two-dimensional ultrasound image data.

In one embodiment, that the information processing unit searches for a spatial frequency corresponding to a maximal value of a distribution indicated by the spatial frequency distribution data in a search range determined in accordance with a pulse width of the transmitted ultrasound, and obtains, as the filter characteristics, characteristics for suppressing a level of the distribution indicated by the spatial frequency distribution data in a spatial frequency band including the spatial frequency that is searched for.

In one embodiment, that the information processing unit changes the filter characteristics in a case in which the maximal value of the distribution indicated by the spatial frequency distribution data does not satisfy a predetermined condition for the spatial frequency distribution data acquired from the two-dimensional ultrasound image data on which the filter processing has been performed.

In one embodiment, the information processing unit generates the two-dimensional ultrasound image data in sequence with elapse of time, and performs the filter processing, which is the same as the filter processing on the two-dimensional ultrasound image data of one frame generated earlier, on the two-dimensional ultrasound image data of one frame generated later.

In one embodiment, the information processing unit performs the filter processing in which the filter characteristics are changed on the two-dimensional ultrasound image data of one frame generated much later in a case in which a condition related to an artifact is not satisfied for the two-dimensional ultrasound image data of the one frame generated later.

In one embodiment, the two-dimensional ultrasound image data is obtained by transmitting and receiving the ultrasound while performing scanning with a transmission beam of the ultrasound at a predetermined scanning interval, and the information processing unit performs the filter processing on the two-dimensional ultrasound image data in a case in which the scanning interval exceeds a predetermined scanning interval threshold value.

In one embodiment, that the scanning interval threshold value is determined based on the characteristics of the transmitted ultrasound.

In one embodiment, the filter characteristics are characteristics for making an attenuation amount in a spatial frequency band corresponding to a predetermined structure be equal to or less than an attenuation amount limit value.

In one embodiment, the two-dimensional ultrasound image data is data indicating a B-mode image on which a blood flow image is superimposed.

According to the aspect of the present disclosure, it is possible to suppress the artifact caused by the pixel value undulating in the depth direction of the ultrasound image.

### BRIEF DESCRIPTION OF THE DRAWINGS

FIG. 1 is a diagram showing a configuration of an ultrasound diagnostic apparatus according to an embodiment of the present disclosure.

FIG. 2 is a diagram showing an example of a positional relationship between a transmission beam and a plurality of reception beams in a case in which linear scanning is performed.

FIG. 3 is a diagram showing an example of a positional relationship between a transmission beam and a plurality of reception beams in a case in which sector scanning is performed.

FIG. 4 is a diagram schematically showing a B-mode image and speckles appearing in the B-mode image.

FIG. 5 is a diagram showing an outline of a spatial frequency distribution.

FIG. 6 is a diagram showing an outline of a transmission pulse transmitted from an ultrasound probe.

FIG. 7 is a diagram showing a spatial frequency distribution in which a lower limit spatial frequency and an upper limit spatial frequency are shown.

FIG. 8 is a diagram showing low-pass filter characteristics.

FIG. 9 is a diagram showing band-stop filter characteristics.

FIG. 10 is a diagram showing notch filter characteristics.

FIG. 11 is a flowchart of processing of determining unnecessary component suppression filter characteristics and performing filter processing on B-mode image data.

FIG. 12 is a diagram showing a plurality of types of spatial frequency distributions.

FIG. 13 is a diagram showing an outline of a spatial frequency distribution in a case in which an image of a structure appears in the B-mode image, and the unnecessary component suppression filter characteristics.

FIG. 14 is a flowchart of processing of determining the unnecessary component suppression filter characteristics and performing the filter processing on the B-mode image data.

FIG. 15 is a diagram showing an outline of a spatial frequency distribution obtained for one sample line data in the B-mode image data of a certain frame.

FIG. 16 is a flowchart of processing of determining the unnecessary component suppression filter characteristics via adaptive filter processing and performing the filter processing on the B-mode image data.

FIG. 17 is a diagram showing an example of an image showing the spatial frequency distribution and the unnecessary component suppression filter characteristics along with the B-mode image.

FIG. 18 is a flowchart of processing of determining the unnecessary component suppression filter characteristics and performing the filter processing on the B-mode image data.

FIG. 19 is a diagram showing a configuration of a color Doppler ultrasound diagnostic apparatus according to an application embodiment of the present disclosure.

### DESCRIPTION OF THE PREFERRED EMBODIMENTS

Embodiments of the present disclosure will be described with reference to the respective drawings. The same components shown in a plurality of drawings will be denoted by the same reference numerals, and the description thereof will be omitted. It should be noted that, in the present specification, for a term “P image data” for specifying certain image data, an image indicated by the P image data will be referred to as a P image.

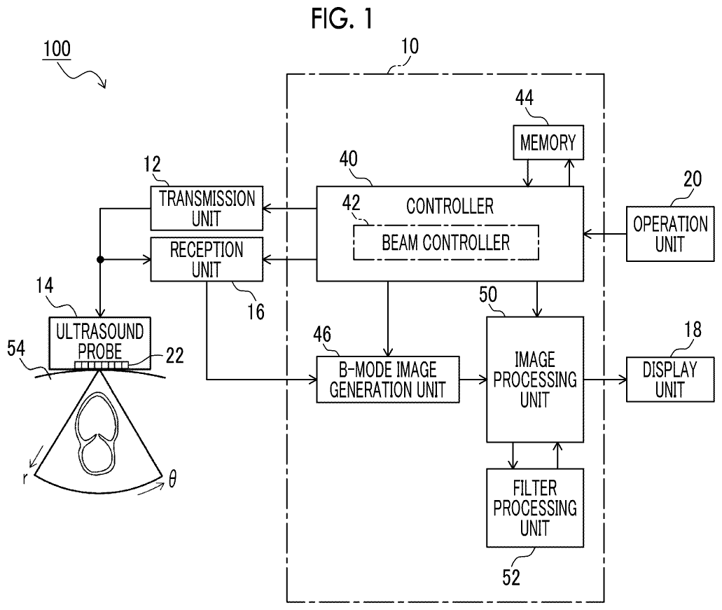

FIG. 1 shows a configuration of an ultrasound diagnostic apparatus 100 according to the embodiment of the present disclosure. The ultrasound diagnostic apparatus 100 comprises an information processing unit 10, a transmission unit 12, an ultrasound probe 14, a reception unit 16, a display unit 18, and an operation unit 20. The information processing unit 10 comprises a controller 40, a memory 44, a B-mode image generation unit 46, an image processing unit 50, and a filter processing unit 52, and constitutes a three-dimensional ultrasound image processing apparatus that executes processing related to an ultrasound image.

The information processing unit 10 may be configured by one or a plurality of processors that execute a program to realize functions of the controller 40, the B-mode image generation unit 46, the image processing unit 50, and the filter processing unit 52. The program may be stored in the memory 44.

The controller 40 performs overall control on the ultrasound diagnostic apparatus 100. The operation unit 20 includes a keyboard, a mouse, a lever, a button, and the like, and outputs information related to an operation of a user to the controller 40. In a case in which the display unit 18 includes a touch panel on a display screen, the operation unit 20 may include the touch panel. The controller 40 may control the ultrasound diagnostic apparatus 100 in accordance with an operation on the operation unit 20.

The operation of the ultrasound diagnostic apparatus 100 will be described. The ultrasound probe 14 is in a state of being in contact with a surface of the subject 54. The ultrasound probe 14 comprises a plurality of oscillating elements 22. The transmission unit 12 outputs a transmission signal to each oscillating element 22 of the ultrasound probe 14 based on control via a beam controller 42 provided in the controller 40. As a result, the ultrasound is transmitted from the ultrasound probe 14. The beam controller 42 forms a transmission beam in the ultrasound probe 14 to scan the subject 54 with the transmission beam by controlling the transmission unit 12. That is, the transmission unit 12 adjusts a delay time or a level of each transmission signal in accordance with the control of the beam controller 42, forms the transmission beam in the ultrasound probe 14, and scans the subject 54 with the transmission beam.

In a case in which the ultrasound reflected in the subject 54 is received by each oscillating element 22 of the ultrasound probe 14, each oscillating element 22 outputs an electric signal corresponding to the received ultrasound to the reception unit 16. The reception unit 16 performs processing, such as amplification, detection, and frequency band limitation, on the reception signal output from each oscillating element 22 in accordance with the control of the beam controller 42. The reception unit 16 further performs phasing addition on the reception signals output from the respective oscillating elements 22 to generate a post-phasing reception signal. As a result, the post-phasing reception signals in which the phases are adjusted and added such that the reception signals based on the ultrasound received from a specific direction reinforce each other are generated, and a reception beam is formed in the specific direction. The reception unit 16 outputs the post-phasing reception signal to the B-mode image generation unit 46.

The B-mode image generation unit 46 generates the B-mode image data based on the post-phasing reception signal obtained in each reception beam direction, and outputs the B-mode image data to the image processing unit 50. The B-mode image data based on one scanning with the transmission beam and the reception beam is image data for one frame, and corresponds to one B-mode image.

The ultrasound probe 14 has a two-dimensional array structure, and swings a scanning surface scanned with the transmission beam and the reception beam in a direction intersecting the scanning surface, for example, the azimuthal angle direction of the depth direction. The ultrasound probe 14 swings, for example, the scanning surface in the azimuthal angle direction of the depth direction by a predetermined step width each time the B-mode image data of one scanning surface is generated. The B-mode image generation unit 46 generates the B-mode image data as two-dimensional ultrasound image data of one scanning surface each time the ultrasound probe 14 swings the scanning surface by the step width, and outputs the B-mode image data to the image processing unit 50. The image processing unit 50 generates three-dimensional ultrasound image data composed of the B-mode image data (two-dimensional ultrasound image data) of a plurality of frames acquired at different positions in the subject 54. The three-dimensional ultrasound image data is data representing each pixel value of a plurality of voxels (pixels) arranged in three axial directions.

The image processing unit 50 selects the B-mode image data of one frame in the B-mode image data of the plurality of frames based on the operation of the operation unit 20, generates a video signal for displaying an image indicated by the selected B-mode image data, and outputs the video signal to the display unit 18. The display unit 18 displays the B-mode image based on the video signal. In addition, the image processing unit 50 may generate volume rendering image data in which the three-dimensional ultrasound image is represented as a two-dimensional image in a stereoscopic manner. The image processing unit 50 generates the video signal for displaying the volume rendering image, to output the video signal to the display unit 18. The display unit 18 displays the volume rendering image based on the video signal.

It should be noted that, in the ultrasound diagnostic apparatus 100 according to the present embodiment, the three-dimensional ultrasound image data representing the plurality of voxels arranged in the three axial directions is acquired by swinging the scanning surface via the ultrasound probe 14. In addition to such processing, processing of transporting the ultrasound probe 14 in a linear or curved manner to acquire the three-dimensional ultrasound image data may be executed.

For example, it is assumed that the plurality of oscillating elements 22 are two-dimensionally arranged along a major axis direction and a minor axis direction of a substantially rectangular surface of the ultrasound probe 14 in contact with the subject 54. In this case, a surface including an imaginary major axis extending in the major axis direction may be defined as the scanning surface, and the ultrasound probe 14 may be transported in the minor axis direction in a linear manner. The ultrasound probe 14 is transported in the minor axis direction by a predetermined step width, for example, each time the B-mode image data of one frame is generated. The B-mode image generation unit 46 generates the B-mode image data as the two-dimensional ultrasound image data of one frame each time the ultrasound probe 14 is transported by the step width, and outputs the B-mode image data to the image processing unit 50. The image processing unit 50 generates three-dimensional ultrasound image data composed of the B-mode image data (two-dimensional ultrasound image data) of a plurality of frames acquired at different positions in the subject 54.

In order to maintain a frame rate of the B-mode image data generated by the ultrasound diagnostic apparatus 100 at a certain magnitude, it is necessary to reduce a time for scanning the transmission beam on one scanning surface. Therefore, the number of transmission beams per frame in a case of acquiring the three-dimensional ultrasound image data is smaller than the number of transmission beams in a case of generating the B-mode image data only on one scanning surface. Further, a plurality of reception beams are formed for one transmission beam, the B-mode image data is generated based on a plurality of reception signals obtained from the plurality of reception beams, that is, signals obtained by parallel reception phasing.

The parallel reception phasing will be described. The reception unit 16 forms a plurality of (M) reception beams for one transmission of the ultrasound pulse. That is, the reception unit 16 performs parallel reception phasing on the plurality of reception signals output from the plurality of oscillating elements 22 to form the M reception beams. In a case in which linear scanning with the transmission beam is performed, the reception unit 16 forms the M reception beams having the same direction as the transmission beam direction and arranged parallel to each other. In a case in which sector scanning with the transmission beam is performed, the reception unit 16 forms the M reception beams arranged at equal azimuthal angle intervals. Here, the azimuthal angle refers to an angle that defines a direction as seen from the center of the sector scanning.

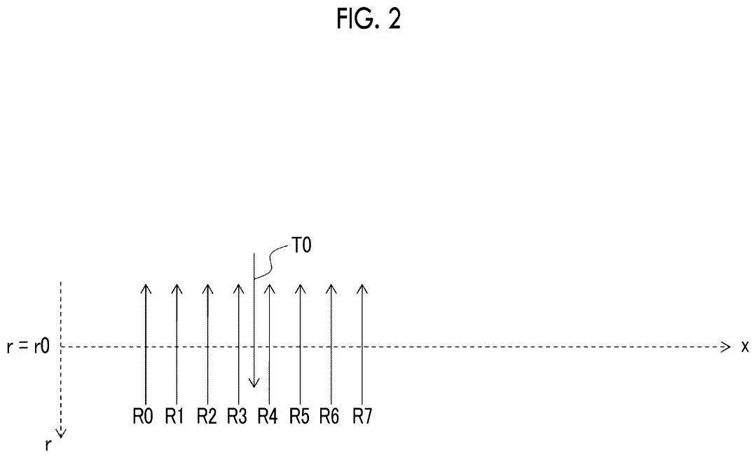

FIG. 2 shows an example of a positional relationship between a transmission beam T0 and reception beams R0 to R7 in a case in which the linear scanning is performed. As shown in FIG. 2, the transmission beam T0 is formed in a depth direction (r-axis positive direction). In this example, eight (M=8) reception beams R0 to R7 arranged at equal intervals and directed in an r-axis negative direction are formed at a position at which a depth is r=r0 with the transmission beam T0 as a center.

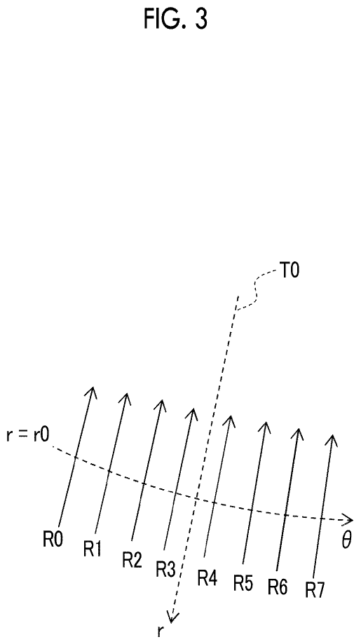

FIG. 3 shows an example of a positional relationship between the transmission beam T0 and the reception beams R0 to R7 in a case in which the sector scanning is performed. In FIG. 3, the r-axis positive direction is determined in the depth direction, and a θ-axis is determined in an angle direction in which the transmission beam swings. FIG. 3 shows a state in which eight (M=8) reception beams R0 to R7 arranged at equal angle intervals and directed in the r-axis negative direction are formed at a position having a depth of r=r0 with the transmission beam T0 as a center.

The reception unit 16 generates the post-phasing reception signal for each of the M reception beams, to output the post-phasing reception signal generated for each of the M reception beams to the B-mode image generation unit 46. The B-mode image generation unit 46 generates the B-mode image data of a region in which M reception beams are disposed in each direction of the scanned transmission beam.

In the B-mode image indicated by the B-mode image data generated by the parallel reception phasing, spot-like speckles extending in the scanning direction may be caused by the pixel value undulating in the depth direction as well as the scanning direction of the transmission beam. Here, the scanning direction is a direction perpendicular to the transmission beam in the linear scanning and is an azimuthal angle direction in the sector scanning. Such speckles may cause a stripe pattern extending in the scanning direction to appear as an artifact. This stripe-like artifact tends to be more remarkable as a scanning interval is larger.

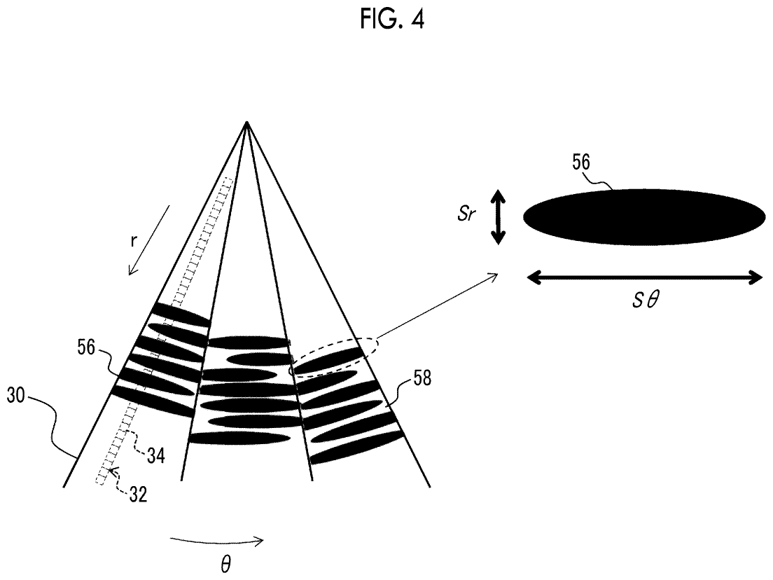

FIG. 4 schematically shows a B-mode image 30 obtained by the sector scanning and speckles 56 appearing in the B-mode image 30. As shown in FIG. 4, the stripe pattern extending in the scanning direction (azimuthal angle direction) appears as the artifact due to gaps 58 among a plurality of speckles 56 extending in the scanning direction. As the scanning interval is larger, the shape of one speckle 56 extending in the scanning direction is more apparent, and the stripe pattern is more remarkable.

Therefore, in the ultrasound diagnostic apparatus 100 according to the present embodiment, the filter processing unit 52 determines filter characteristics for suppressing the stripe-like artifact based on the B-mode image data. The filter processing unit 52 performs the filter processing based on the filter characteristics, on the B-mode image data to generate filter-processed B-mode image data. The image processing unit 50 displays, on the display unit 18, a filter-processed B-mode image or the volume rendering image based on the filter-processed B-mode images of a plurality of frames. Hereinafter, specific processing is shown.

The image processing unit 50 outputs the B-mode image data to the filter processing unit 52. The filter processing unit 52 extracts depth direction line data representing each pixel value of a plurality of pixels arranged in the depth direction for each of a plurality of azimuthal angle directions from the B-mode image data. In FIG. 4, one set of depth direction line data 32 and pixels 34 arranged in the depth direction are conceptually shown on the B-mode image 30.

The filter processing unit 52 selects one set of a plurality of sets of depth direction line data 32 generated for the plurality of azimuthal angle directions as sample line data. One set of sample line data corresponds to one set of depth direction line data 32 corresponding to one azimuthal angle direction. The filter processing unit 52 performs spatial Fourier transformation on the sample line data to obtain the spatial frequency distribution data.

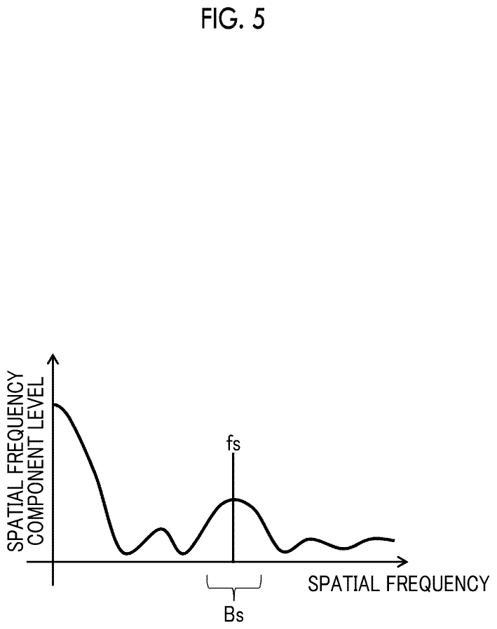

FIG. 5 shows an outline of a distribution (spatial frequency distribution) indicated by the spatial frequency distribution data obtained for the sample line data. A horizontal axis represents a spatial frequency, and a vertical axis represents a level of a spatial frequency component (spatial frequency component level). A speckle spatial frequency fs shown in FIG. 5 corresponds to the maximal value appearing based on the speckles. In addition, a speckle spatial frequency band Bs is a spatial frequency band occupied by the speckles.

The filter processing unit 52 searches for the speckle spatial frequency fs corresponding to the maximal value of the spatial frequency distribution of the sample line data, to define the speckle spatial frequency band Bs including the speckle spatial frequency fs. The filter processing unit 52 further performs the filter processing of suppressing the spatial frequency component of the speckle spatial frequency band Bs on the B-mode image data. Specifically, the filter processing unit 52 performs the filter processing of suppressing the spatial frequency component of the speckle spatial frequency band Bs on the plurality of sets of depth direction line data 32 corresponding to the plurality of azimuthal angle directions extending over the entire region of the B-mode image. As a result, the filter processing unit 52 generates the filter-processed B-mode image data to output the filter-processed B-mode image data to the image processing unit 50. The filter-processed B-mode image data is composed of a plurality of sets of filter-processed depth direction line data obtained by performing the filter processing on the plurality of sets of depth direction line data 32 corresponding to the plurality of azimuthal angle directions extending over the entire region of the B-mode image.

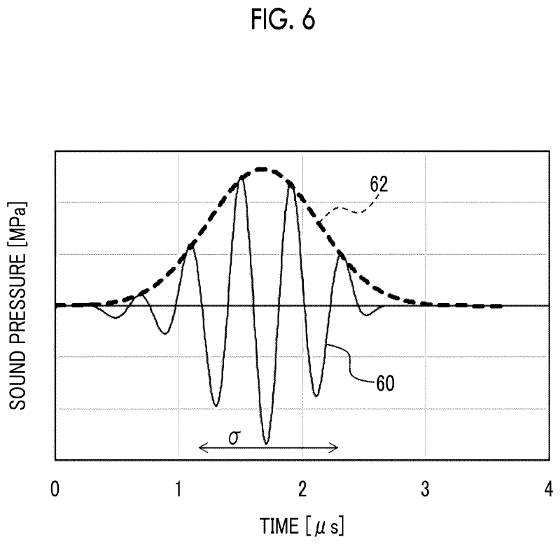

The processing of searching for the speckle spatial frequency fs, defining the speckle spatial frequency band Bs, and obtaining the filter characteristics will be described. FIG. 6 schematically shows a transmission pulse 60 transmitted from the oscillating element 22 to generate the B-mode image data. A horizontal axis represents a time, and a vertical axis represents a sound pressure. FIG. 6 shows a pulse width σ as one feature value indicating characteristics of transmitted ultrasound. The pulse width σ is defined, for example, as a time length in which the sound pressure is half of the maximum value for an envelope 62 of the transmission pulse 60. An average value Sr [mm] of widths of the speckles 56 in the depth direction shown in FIG. 4 is approximately represented as (Expression 1) using the pulse width σ [sec] of the transmission pulse and a sound velocity c [mm/sec] in the subject 54.

$$Sr = 2.51 \times σ \times c \qquad ( Expression 1 )$$

It is known that the speckle spatial frequency fs is close to a reference spatial frequency fs0 obtained by multiplying the sound velocity c by a reciprocal of Sr, as shown in (Expression 2).

$$fs 0 = c \times 1 / Sr = 1 / ( 2.51 σ ) \qquad ( Expression 2 )$$

The filter processing unit 52 acquires the spatial frequency component level corresponding to the spatial frequency while increasing the spatial frequency in sequence from the reference spatial frequency fs0 by a step width δ within the search range including the reference spatial frequency fs0 (within the spatial frequency band determined in accordance with the pulse width of the transmitted ultrasound). In addition, the filter processing unit 52 acquires the spatial frequency component level corresponding to the spatial frequency while decreasing the spatial frequency in sequence from the reference spatial frequency fs0 by the step width δ. The filter processing unit 52 determines the spatial frequency at which the spatial frequency component level is maximal, as the speckle spatial frequency fs.

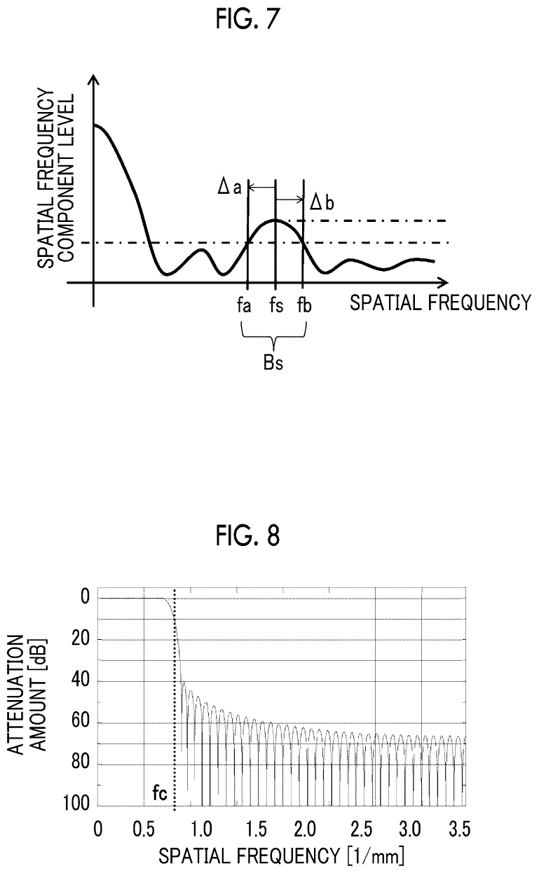

The filter processing unit 52 determines a spatial frequency higher than the speckle spatial frequency fs by a predetermined upper limit spatial frequency width Δb as an upper limit spatial frequency fb of the speckle spatial frequency band Bs, as shown in FIG. 7. In addition, the filter processing unit 52 determines a spatial frequency lower than the speckle spatial frequency fs by a predetermined lower limit frequency width Δa as a lower limit spatial frequency fa of the speckle spatial frequency band Bs.

The filter processing unit 52 forms a band suppression filter in which the speckle spatial frequency band Bs equal to or higher than the lower limit spatial frequency fa and equal to or lower than the upper limit spatial frequency fb is a suppression band width. The band suppression filter has, for example, unnecessary component suppression filter characteristics for suppressing the level by 6 dB or higher in the frequency band equal to or higher than the lower limit spatial frequency fa and equal to or lower than the upper limit spatial frequency fb. The unnecessary component suppression filter characteristics may be characteristics for attenuating the level by 6 dB or higher, preferably 20 dB or higher, at the speckle spatial frequency fs.

FIG. 8 shows low-pass filter characteristics as an example of the unnecessary component suppression filter characteristics. An attenuation amount in a case in which the spatial frequency is 0 is 0 dB, and an attenuation amount of 6 dB or higher is realized in a frequency band equal to or higher than a cutoff spatial frequency fc. The cutoff spatial frequency fc is set to a spatial frequency lower than the lower limit spatial frequency fa. It should be noted that the attenuation amount is a value on a decibel scale that is increased in a positive direction as the attenuation is increased.

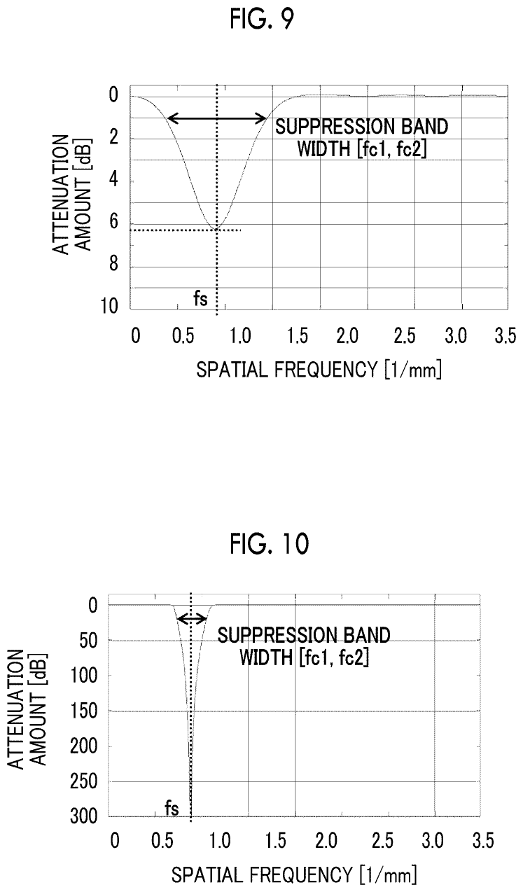

FIG. 9 shows band-stop filter characteristics as an example of the unnecessary component suppression filter characteristics. An attenuation amount in a case in which the spatial frequency is 0 and is infinite is set to 0 dB, and an attenuation amount of 6 dB or higher is realized in the spatial frequency band equal to or higher than a low-band cutoff spatial frequency fc1 and equal to or lower than a high-band cutoff spatial frequency fc2. For example, the low-band cutoff spatial frequency fc1 is set to the lower limit spatial frequency fa, and the high-band cutoff spatial frequency fc2 is set to the upper limit spatial frequency fb.

FIG. 10 shows notch filter characteristics as an example of the unnecessary component suppression filter characteristics. The notch filter characteristics are special band-stop filter characteristics. In the notch filter characteristics, a notch in which the attenuation amount is theoretically infinite appears in an attenuation band. For example, the low-band cutoff spatial frequency fc1 is set to the lower limit spatial frequency fa, and the high-band cutoff spatial frequency fc2 is set to the upper limit spatial frequency fb.

The filter processing unit 52 performs the filter processing based on the unnecessary component suppression filter characteristics for each of the plurality of sets of depth direction line data 32 corresponding to the plurality of azimuthal angle directions extending over the entire region of the B-mode image, and generates the plurality of sets of filter-processed depth direction line data corresponding to the plurality of azimuthal angle directions extending over the entire region of the B-mode image. The filter processing unit 52 forms the filter-processed B-mode image data based on the plurality of sets of filter-processed depth direction line data, and outputs the filter-processed B-mode image data to the image processing unit 50. The image processing unit 50 generates the video signal for displaying the filter-processed B-mode image, to output the video signal to the display unit 18. The display unit 18 displays the filter-processed B-mode image based on the video signal. In addition, the image processing unit 50 may generate a video signal for displaying the volume rendering image based on the filter-processed B-mode image of the plurality of frames, and may output the video signal to the display unit 18. In this case, the display unit 18 displays the volume rendering image based on the video signal.

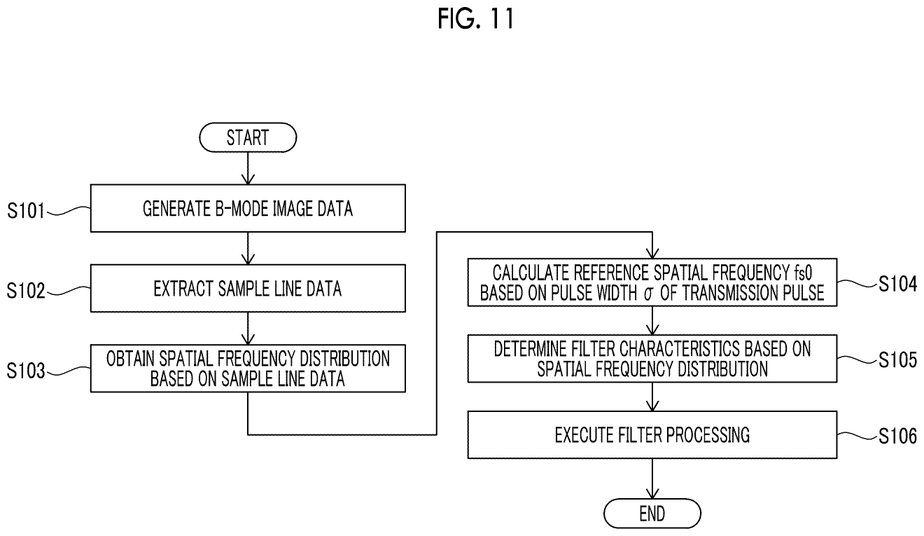

FIG. 11 shows a flowchart of processing of determining the unnecessary component suppression filter characteristics and performing the filter processing on the B-mode image data. In FIG. 11, the term “unnecessary component suppression filter characteristics” is referred to as “filter characteristics”, and the expression is simplified. The B-mode image generation unit 46 generates the B-mode image data (S101). The filter processing unit 52 extracts the sample line data from the B-mode image data (S102), and performs the spatial Fourier transformation on the sample line data to obtain the spatial frequency distribution (S103). The filter processing unit 52 calculates the reference spatial frequency fs0 based on the pulse width σ of the transmission pulse in accordance with (Expression 2) (S104). The filter processing unit 52 searches for the speckle spatial frequency fs by using the reference spatial frequency fs0, to determine the unnecessary component suppression filter characteristics (S105). The filter processing unit 52 performs the filter processing on the B-mode image data based on the determined unnecessary component suppression filter characteristics, to generate the filter-processed B-mode image data (S106).

With such processing, as the characteristics of the transmitted ultrasound, the reference spatial frequency fs0 is obtained in accordance with (Expression 2) based on the pulse width o of the transmission pulse. Then, the speckle spatial frequency fs at which the level is maximal in the spatial frequency distribution is searched for on the high-band side and the low-band side of the reference spatial frequency fs0. Further, the spatial frequency higher than the speckle spatial frequency fs by the predetermined upper limit spatial frequency width Δb is determined as the upper limit spatial frequency fb, the spatial frequency lower than the speckle spatial frequency fs by the predetermined lower limit frequency width Δa is determined as the lower limit spatial frequency fa, and the unnecessary component suppression filter characteristics in the filter processing unit 52 are determined. As a result, the processing of determining the unnecessary component suppression filter characteristics for suppressing the stripe-like artifact is quickly performed.

In the processing, the filter processing unit 52 determines the spatial frequency higher than the speckle spatial frequency fs by the predetermined upper limit spatial frequency width Δb as the upper limit spatial frequency fb, and determines the spatial frequency lower than the speckle spatial frequency fs by the predetermined lower limit frequency width Δa as the lower limit spatial frequency fa. As described above, instead of the processing of determining the spatial frequencies separated from the speckle spatial frequency fs by a certain value as the upper limit spatial frequency fb and the lower limit spatial frequency fa, the following processing may be executed.

That is, the filter processing unit 52 may determine the spatial frequency on the low-band side at which the level of the spatial frequency distribution is decreased by a predetermined ratio with respect to the maximal value of the speckle spatial frequency fs of the spatial frequency distribution, as the lower limit spatial frequency fa. In addition, the filter processing unit 52 may determine the spatial frequency on the high-band side at which the level of the spatial frequency distribution is decreased by a predetermined ratio, as the upper limit spatial frequency fb. As will be described below, the filter processing unit 52 may execute processing of adaptively determining the lower limit spatial frequency fa and the upper limit spatial frequency fb.

The filter processing unit 52 selects a plurality of (K) sets among a plurality of sets of depth direction line data 32 generated for the plurality of azimuthal angle directions as the K sets of sample line data. The filter processing unit 52 performs the spatial Fourier transformation on each of the K sets of sample line data to obtain K types of spatial frequency distributions. The filter processing unit 52 searches for the speckle spatial frequency fs for each of the K types of spatial frequency distributions via the same processing as the processing based on the one set of sample line data. The filter processing unit 52 obtains the minimum speckle spatial frequency fs among the K speckle spatial frequencies fs obtained from the K types of spatial frequency distributions as the lower limit spatial frequency fa, and obtains the maximum speckle spatial frequency fs as the upper limit spatial frequency fb.

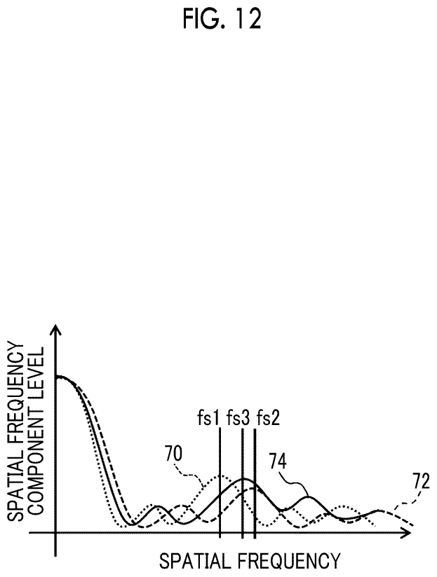

FIG. 12 shows a spatial frequency distribution 70 in which a minimum speckle spatial frequency fs1 is obtained, a spatial frequency distribution 72 in which a maximum speckle spatial frequency fs2 is obtained, and a spatial frequency distribution 74 in which a speckle spatial frequency fs3 between the speckle spatial frequency fs1 and the speckle spatial frequency fs2 is obtained. The filter processing unit 52 sets the unnecessary component suppression filter characteristics in the filter processing unit 52 by setting the minimum speckle spatial frequency fs1 as the lower limit spatial frequency fa and the maximum speckle spatial frequency fs2 as the upper limit spatial frequency fb.

The filter processing unit 52 may obtain one spatial frequency distribution by obtaining a statistical value such as an average value, a median value, a maximum value, and a minimum value of K spatial frequency component levels at each spatial frequency, from the K types of spatial frequency distributions obtained from the K sets of sample line data. The filter processing unit 52 may execute the same processing as the processing based on the one set of sample line data for one spatial frequency distribution obtained from the K types of spatial frequency distributions. That is, the filter processing unit 52 may search for the speckle spatial frequency fs for one spatial frequency distribution obtained from the K types of spatial frequency distributions, to obtain the lower limit spatial frequency fa and the upper limit spatial frequency fb.

An image of a structure of biological tissue, such as a blood vessel, a heart wall, or a heart valve, may appear in the B-mode image. In this case, in the filter-processed B-mode image in which the stripe-like artifact is suppressed, the structure may be difficult to see. The spatial frequency component included in the sample line data by the structure may be lower than the speckle spatial frequency. Therefore, the filter processing unit 52 may execute processing of limiting the attenuation amount in the spatial frequency band corresponding to the structure.

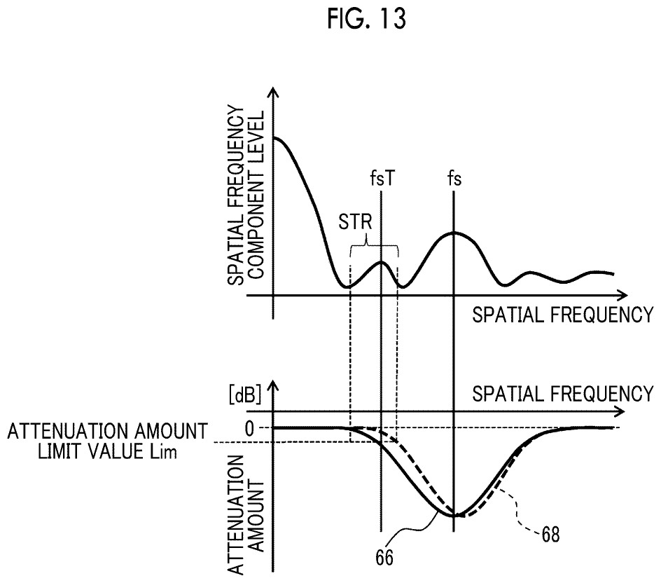

An upper part of FIG. 13 shows an outline of the spatial frequency distribution in a case in which the image of the structure appears in the B-mode image. In this spatial frequency distribution, the maximal value appears in the speckle spatial frequency fs, and the maximal value also appears in a structure spatial frequency fsT corresponding to the structure. The controller 40 reads the structure spatial frequency fsT and a spatial frequency band STR including the structure spatial frequency fsT in accordance with the operation of the operation unit 20 performed by the user. The structure spatial frequency fsT and the spatial frequency band STR may be stored in the memory 44 in advance and read by the controller 40. The filter processing unit 52 determines the unnecessary component suppression filter characteristics such that the attenuation amount is equal to or less than a predetermined attenuation amount limit value Lim in the spatial frequency band STR including the structure spatial frequency fsT, based on the control of the controller 40.

A lower part of FIG. 13 shows an example of unnecessary component suppression filter characteristics 66 in which the attenuation amount is not limited in the spatial frequency band STR and an example of unnecessary component suppression filter characteristics 68 in which the attenuation amount is limited in the spatial frequency band STR. By performing, via the filter processing unit 52, the filter processing using the unnecessary component suppression filter characteristics 68 on the B-mode image data, the stripe-like artifact that appears in the filter-processed B-mode image is suppressed, and the structure is clearly represented.

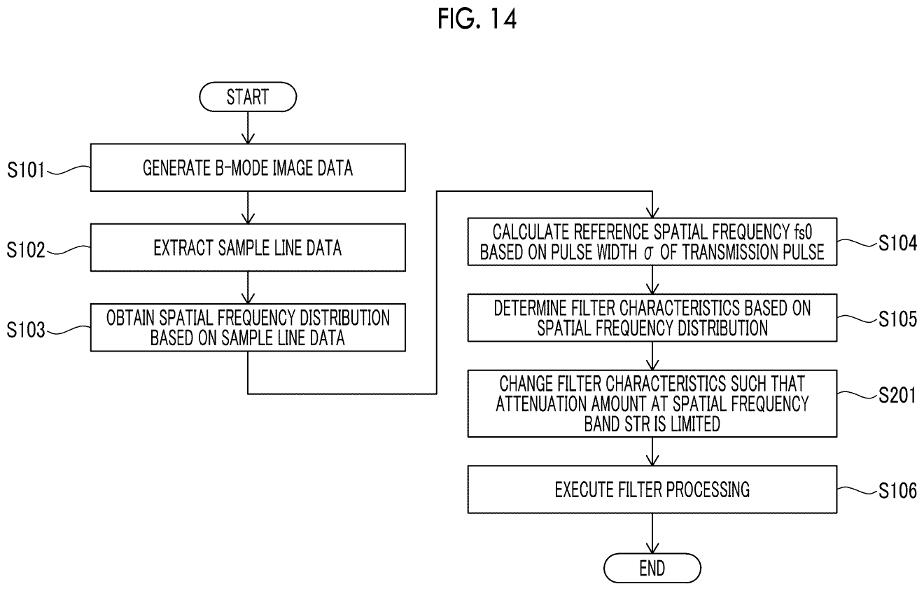

FIG. 14 shows a flowchart of processing of determining the unnecessary component suppression filter characteristics and performing the filter processing on the B-mode image data. This flowchart is different from the flowchart shown in FIG. 11 in that step S201 is added between step S105 and step S106. In FIG. 14, the term “unnecessary component suppression filter characteristics” is referred to as “filter characteristics”, and the expression is simplified. The filter processing unit 52 searches for the speckle spatial frequency fs using the reference spatial frequency fs0, to determine the unnecessary component suppression filter characteristics (S105), and then changes the unnecessary component suppression filter characteristics such that the attenuation amount in the spatial frequency band STR is equal to or less than the attenuation amount limit value Lim (S201). The filter processing unit 52 performs the filter processing on the B-mode image data based on the unnecessary component suppression filter characteristics changed in step S201, to generate the filter-processed B-mode image data (S106).

The B-mode image generation unit 46 outputs the B-mode image data to the image processing unit 50 in sequence with the elapse of time. The image processing unit 50 generates the B-mode image data in sequence with the elapse of time to output the B-mode image data to the filter processing unit 52. The filter processing unit 52 performs the filter processing on each of the B-mode image data output from the image processing unit 50 in sequence with the elapse of time, and outputs the filter-processed B-mode image data to the image processing unit 50 in sequence with the elapse of time.

The filter processing unit 52 may perform the filter processing (hereinafter, also referred to as adaptive filter processing) in which filter characteristics are adaptively changed in accordance with the filter-processed B-mode image data generated in the past, on the B-mode image data output in sequence from the image processing unit 50. Further, the filter processing unit 52 may output the filter-processed B-mode image data to the image processing unit 50 in sequence with the elapse of time.

Here, the adaptive filter processing will be described. In the adaptive filter processing, processing of changing the filter characteristics is repeated in a case in which the maximal value of the distribution indicated by the spatial frequency distribution data does not satisfy a predetermined condition, for the spatial frequency distribution data acquired from the filter-processed B-mode image data. Here, the predetermined condition is a condition for suppressing the stripe-like artifact.

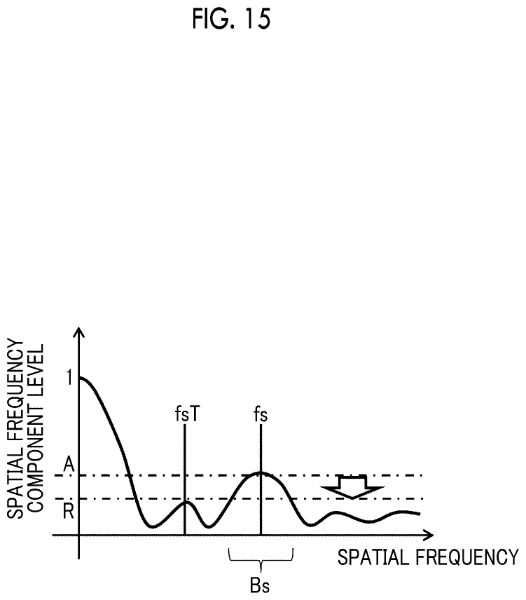

FIG. 15 shows an outline of the spatial frequency distribution obtained for one sample line data in the filter-processed B-mode image data of a certain frame. The level of the component having the spatial frequency of 0 is standardized to 1, and the maximal value at the speckle spatial frequency fs is A. In a case in which a target value of the level at the speckle spatial frequency fs is R, and R<A is satisfied, the filter processing unit 52 adjusts and changes the unnecessary component suppression filter characteristics such that the attenuation amount at the speckle spatial frequency fs is 20 log(A/R) [dB] or more. Here, log is a logarithmic function with 10 as a base, and the attenuation amount is a value that is larger in the positive direction as the attenuation is larger.

The filter processing unit 52 performs the filter processing based on the unnecessary component suppression filter characteristics changed based on the earlier B-mode image data, on the B-mode image data of one frame output later from the image processing unit 50, and outputs the filter-processed B-mode image data to the image processing unit 50. In a case in which R<A is satisfied again in the spatial frequency distribution obtained for one sample line data in the filter-processed B-mode image data of one frame output to the image processing unit 50, the filter processing unit 52 changes the unnecessary component suppression filter characteristics such that the attenuation amount at the speckle spatial frequency fs is 20 log(A/R) or more.

In this way, the filter processing unit 52 sets the unnecessary component suppression filter characteristics based on the earlier data in the filter-processed B-mode image data generated in sequence with the elapse of time, and performs the filter processing based on the determined filter characteristics, on the B-mode image data of the next frame. The filter processing unit 52 executes such processing in sequence on the B-mode image data generated in sequence with the elapse of time, so that the unnecessary component suppression filter characteristics converge to ideal characteristics.

From another viewpoint, the filter processing unit 52 executes the following processing. That is, the filter processing unit 52 performs the same filter processing as the filter processing on the B-mode image data of one frame generated earlier, on the B-mode image data of one frame generated later. In a case in which the condition related to the artifact is not satisfied for the B-mode image data of one frame generated later, the filter processing unit 52 performs the filter processing in which the filter characteristics are changed, on the B-mode image data of the frame generated much later. The condition related to the artifact is, for example, a condition in which the maximal value A and the target value R at the speckle spatial frequency fs satisfy A≤R for the spatial frequency distribution obtained for one sample line data.

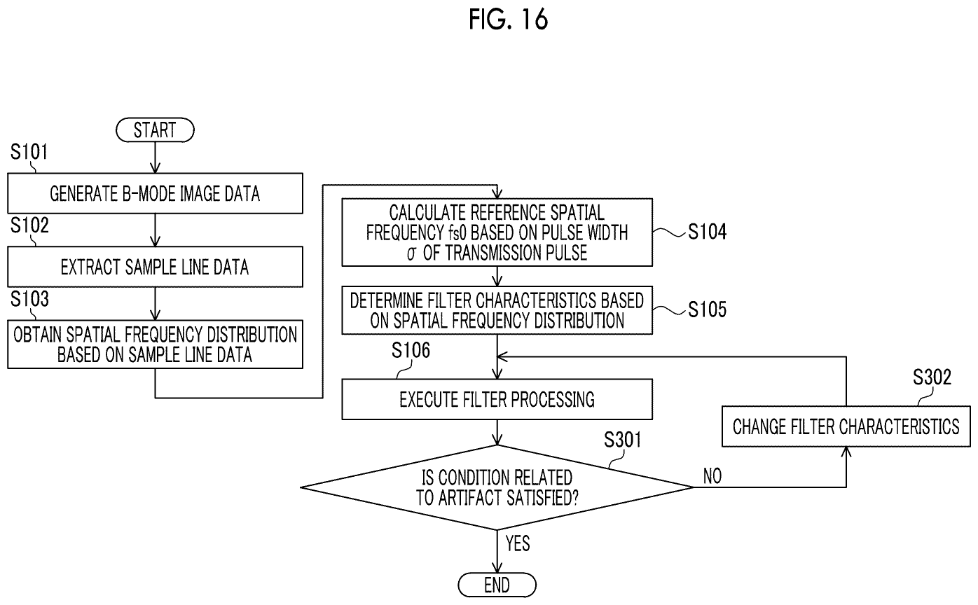

FIG. 16 is a flowchart of processing of determining the unnecessary component suppression filter characteristics via the adaptive filter processing and performing the filter processing on the B-mode image data. This flowchart is different from the flowchart shown in FIG. 11 in that steps S301 and S302 are added after step S106. In FIG. 16, the term “unnecessary component suppression filter characteristics” is referred to as “filter characteristics”, and the expression is simplified.

The filter processing unit 52 searches for the speckle spatial frequency fs by using the reference spatial frequency fs0, to determine the unnecessary component suppression filter characteristics (S105). The filter processing unit 52 performs the filter processing on the B-mode image data based on the determined unnecessary component suppression filter characteristics, to generate the filter-processed B-mode image data (S106).

The filter processing unit 52 extracts the sample line data from the filter-processed B-mode image data, and determines whether or not the obtained spatial frequency distribution of the sample line data satisfies the condition related to the artifact (S301). In a case in which the condition related to the artifact is satisfied, the filter processing unit 52 terminates the processing while maintaining the unnecessary component suppression filter characteristics.

On the other hand, in a case in which the condition related to the artifact is not satisfied, the filter processing unit 52 changes the unnecessary component suppression filter characteristics (S302), and returns the processing to the processing of step S107. In step S107 from the second time onwards, the filter processing unit 52 performs the filter processing based on the changed unnecessary component suppression filter characteristics, on the filter-processed B-mode image data generated earlier (S106).

With such processing, in steps S301 and S302, the unnecessary component suppression filter characteristics are repeatedly changed for the filter-processed B-mode image data generated in sequence with the elapse of time until the condition related to the artifact is satisfied. As a result, the unnecessary component suppression filter characteristics converge to characteristics in which the condition related to the artifact is satisfied for the filter-processed B-mode image data.

Whether or not the filter processing is performed on the B-mode image data may be determined in accordance with the scanning interval. For example, the filter processing may be performed in a case in which the scanning interval in the azimuthal angle direction exceeds a predetermined scanning interval threshold value. In a case in which the scanning interval exceeds the predetermined scanning interval threshold value, the controller 40 causes the image processing unit 50 and the filter processing unit 52 to execute the filter processing. On the other hand, in a case in which the scanning interval is equal to or less than the scanning interval threshold value, the controller 40 does not cause the image processing unit 50 and the filter processing unit 52 to execute the filter processing on the B-mode image data, and displays the B-mode image based on the B-mode image data on which the filter processing has not been performed or the volume rendering image, on the display unit 18.

The scanning interval threshold value may be determined based on a theoretical value of the magnitude of the speckles 56 in the scanning direction. An average value Sθ [mm] of the widths of the speckles 56 in the scanning direction is approximately represented as (Expression 3). Here, λ is a wavelength [mm] of the ultrasound, z is a measurement depth [mm], and D is an opening width [mm]. The opening width D is a length in which the plurality of oscillating elements 22 provided in the ultrasound probe 14 are arranged on a surface scanned with the ultrasound beam.

$$S θ = 0 \text{.9} λ z / D \qquad ( Expression 3 )$$

In a case in which the width of the speckle 56 in the scanning direction is expressed by the azimuthal angle, a scanning interval threshold value θt [rad] may be obtained in accordance with (Expression 4).

$$θ t \approx arctan ( S θ / z ) = arctan ( 0 \text{.9} λ / D ) \qquad ( Expression 4 )$$

In the ultrasound diagnostic apparatus 100, a man-machine interface that changes the unnecessary component suppression filter characteristics in accordance with the operation of the user may be configured. The man-machine interface may be configured by the image processing unit 50, the display unit 18, and the operation unit 20. That is, the unnecessary component suppression filter characteristics may be set based on the operation of the operation unit 20 performed by the user who refers to the display unit 18. For example, the controller 40 may read the attenuation amounts at the speckle spatial frequency fs, the lower limit spatial frequency fa, the upper limit spatial frequency fb, and the speckle spatial frequency band, the attenuation amounts at the structure spatial frequency fsT and the spatial frequency band STR including the structure spatial frequency fsT, and the like, in accordance with the operation of the operation unit 20. In this case, the image processing unit 50 may display at least one of the spatial frequency distribution obtained for the sample line data or the unnecessary component suppression filter characteristics on the display unit 18, and may present the at least one of the spatial frequency distribution or the unnecessary component suppression filter characteristics to the user. The unnecessary component suppression filter characteristics may be represented by a graph in which a horizontal axis is the spatial frequency and a vertical axis is the attenuation amount, the phase rotation amount, and the like.

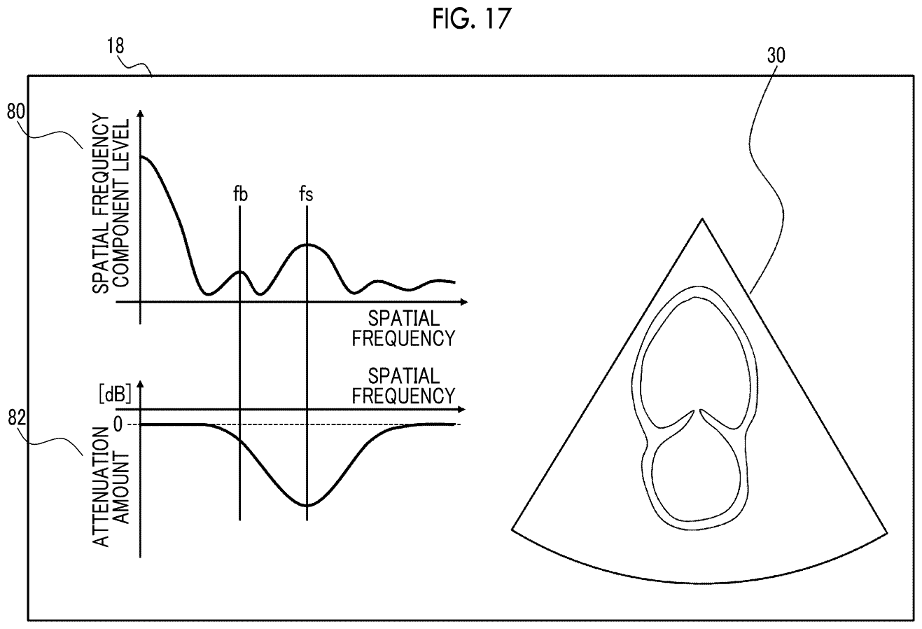

FIG. 17 shows an example of an image showing a spatial frequency distribution 80 and unnecessary component suppression filter characteristics 82 together with the B-mode image. It should be noted that the image processing unit 50 may display the actually applied unnecessary component suppression filter characteristics, the type of the filter (low-pass filter, band-stop filter, notch filter, or the like), and the like on the display unit 18 by text information or the like.

A graphical user interface displayed on the display unit 18 in a case in which the unnecessary component suppression filter characteristics are set may include, for example, a button, an icon, or the like in which “none”, “weak”, “medium”, “strong”, or the like is displayed. In addition, the image processing unit 50 may display a graphical user interface for setting the unnecessary component suppression filter characteristics on the display unit 18 via the operation unit 20.

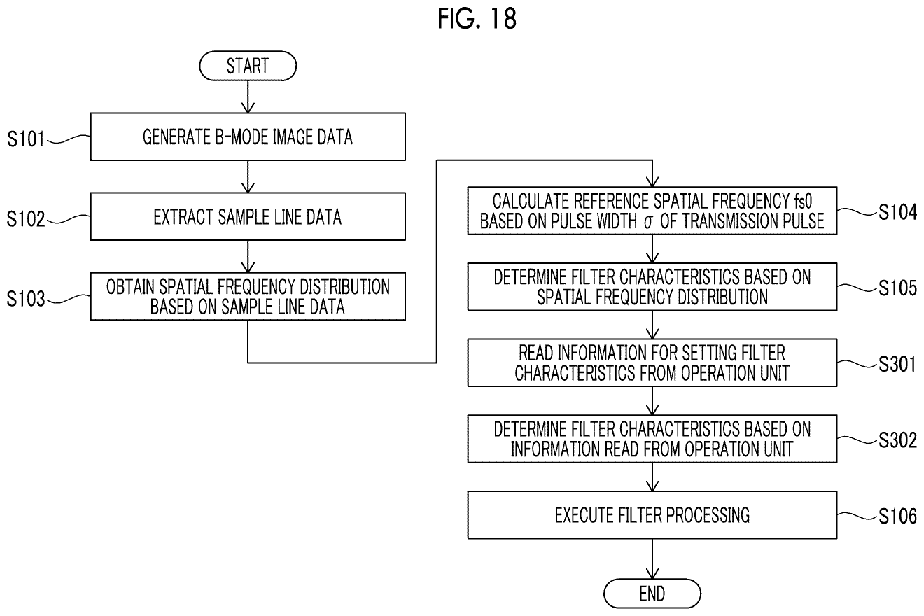

FIG. 18 shows a flowchart of processing of determining the unnecessary component suppression filter characteristics and performing the filter processing on the B-mode image data. This flowchart is different from the flowchart shown in FIG. 11 in that steps S301 and S302 are added between steps S105 and S106. In addition, the term “unnecessary component suppression filter characteristics” is referred to as “filter characteristics”, and the expression is simplified. The filter processing unit 52 searches for the speckle spatial frequency fs by using the reference spatial frequency fs0, to determine the unnecessary component suppression filter characteristics (S105), and further reads information for setting the unnecessary component suppression filter characteristics from the operation unit 20 (S301). The filter processing unit 52 changes the unnecessary component suppression filter characteristics based on the information read from the operation unit 20 (S302). The filter processing unit 52 performs the filter processing on the B-mode image data based on the unnecessary component suppression filter characteristics changed in step S302, to generate the filter-processed B-mode image data (S106).

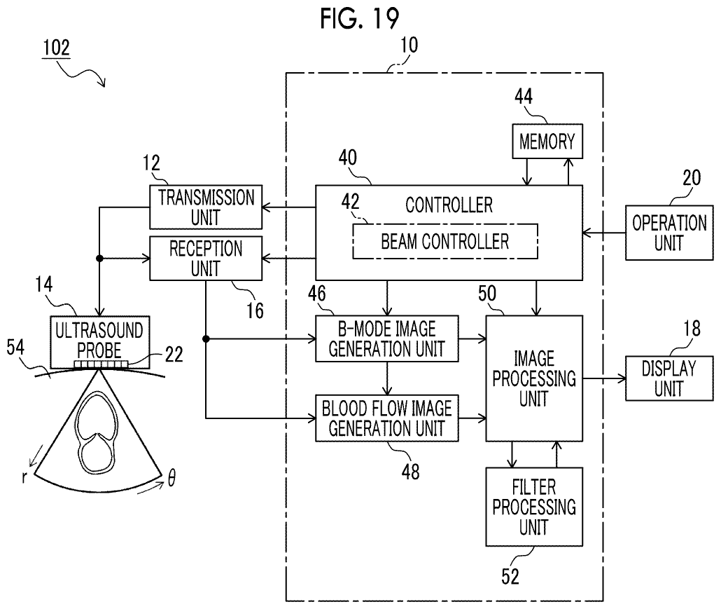

FIG. 19 shows a color Doppler ultrasound diagnostic apparatus 102 according to an application embodiment of the present disclosure. The color Doppler ultrasound diagnostic apparatus 102 is obtained by adding a blood flow image generation unit 48 to the ultrasound diagnostic apparatus 100 shown in FIG. 1. The color Doppler ultrasound diagnostic apparatus 102 executes the measurement in each of the B-mode and the Doppler mode. The color Doppler ultrasound diagnostic apparatus 102 generates the B-mode image data based on the ultrasound reflected by the subject 54 in the operation in the B-mode, and generates the Doppler image data of the subject 54 based on the Doppler shift frequency of the ultrasound received from the subject 54 in the operation in the Doppler mode. Here, the Doppler image data refers to data indicating the Doppler image in which a color indicating a velocity and a direction of the blood flow is attached to the B-mode image. The operation in the B-mode is the same as the operation of generating the B-mode image data via the ultrasound diagnostic apparatus 100 shown in FIG. 1.

The operation in the Doppler mode will be described. In the Doppler mode, the transmission unit 12 outputs the transmission signal to each of the oscillating elements 22 of the ultrasound probe 14 a plurality of (N) times such that ultrasound pulses are transmitted N times at repetitive time intervals T in one transmission beam direction. The reception unit 16 generates N types of post-phasing reception signals for the N ultrasound pulses reflected in the subject 54 and received by the ultrasound probe 14.

The reception unit 16 forms a plurality of (M) reception beams for one transmission of the ultrasound pulse. That is, the reception unit 16 performs parallel reception phasing on the plurality of reception signals output from the plurality of oscillating elements 22 to form the M reception beams. In a case in which linear scanning with the transmission beam is performed, the reception unit 16 forms the M reception beams having the same direction as the transmission beam direction and arranged parallel to each other. In a case in which sector scanning with the transmission beam is performed, the reception unit 16 forms the M reception beams arranged at equal azimuthal angle intervals.

The reception unit 16 generates N types of post-phasing reception signals corresponding to the ultrasound pulses transmitted N times for one transmission beam for each of the M reception beams, and outputs the N types of post-phasing reception signals for each of the M reception beams to the blood flow image generation unit 48.

The blood flow image generation unit 48 obtains a blood flow velocity v(r) in the depth direction at each depth r of each of the M reception beams based on the N types of post-phasing reception signals generated for each of the M reception beams, for example, according to the self-correlation processing described in JP2016-87302A.

The blood flow image generation unit 48 generates blood flow image data based on the blood flow velocity v(r) in the depth direction at each depth r of each of the M reception beams. A blood flow image indicated by the blood flow image data is an image indicating a color in accordance with the blood flow velocity v(r) at a position of each depth r on each reception beam. That is, the blood flow image indicates a distribution of the blood flow velocity v(r) in a region in which the M reception beams are disposed by color. In the blood flow image, for example, a blue color is added to a region in which the blood flows in a direction away from the ultrasound probe 14, a red color is added to a region in which the blood flows in a direction close to the ultrasound probe 14, and the brightness is higher in a region in which the blood flow velocity is higher. The blood flow image generation unit 48 outputs the blood flow image data to the image processing unit 50.

The color Doppler ultrasound diagnostic apparatus 102 executes the measurement in each of the B-mode and the Doppler mode in time division within a time corresponding to one frame, and generates color Doppler image data as data indicating a color Doppler image in which the B-mode image of one frame and the blood flow image of one frame are superimposed on each other.

The B-mode image generation unit 46 generates, for example, the B-mode image data of one scanning surface each time the ultrasound probe 14 swings the scanning surface by the step width, and outputs the B-mode image data to the image processing unit 50. The blood flow image generation unit 48 generates the blood flow image data of one scanning surface each time the ultrasound probe 14 swings the scanning surface by the step width, and outputs the blood flow image data to the image processing unit 50.

The image processing unit 50 generates the Doppler image data acquired at the different positions at intervals of the step widths based on the B-mode image data and the blood flow image data generated each time the ultrasound probe 14 swings the scanning surface by the step width. The image processing unit 50 generates three-dimensional ultrasound image data composed of the Doppler image data of a plurality of frames acquired at different positions in the subject 54.

The image processing unit 50 selects the Doppler image data of one frame in the Doppler image data of the plurality of frames based on the operation of the operation unit 20, generates a video signal for displaying an image indicated by the selected Doppler image data, and outputs the video signal to the display unit 18. The display unit 18 displays the Doppler image based on the video signal. In addition, the image processing unit 50 may generate volume rendering image data in which the three-dimensional ultrasound image is represented as a two-dimensional image in a stereoscopic manner. The image processing unit 50 generates the video signal for displaying the volume rendering image, to output the video signal to the display unit 18. The display unit 18 displays the volume rendering image based on the video signal.

In the B-mode image represented by the B-mode image data generated by the parallel reception phasing, a stripe pattern extending in the scanning direction may appear as the artifact. This stripe-like artifact tends to be more remarkable as a scanning interval is larger

In addition, in order to maintain a frame rate of the B-mode image data generated by the color Doppler ultrasound diagnostic apparatus 102 at a certain magnitude, it is necessary to reduce a time for scanning one scanning surface. Therefore, in the color Doppler ultrasound diagnostic apparatus 102, the scanning interval of the transmission beam in a case in which the measurement in each of the B-mode and the Doppler mode is executed in time division is made larger than the scanning interval in a case in which the measurement is executed only in the B-mode. As a result, the stripe-like artifact appearing in the image displayed on the display unit 18 is remarkable.

Therefore, in the color Doppler ultrasound diagnostic apparatus 102, the image processing unit 50 outputs the B-mode image data to the filter processing unit 52. The filter processing unit 52 performs the filter processing based on the unnecessary component suppression filter characteristics, on the B-mode image data to generate filter-processed B-mode image data, and outputs the filter-processed B-mode image data to the image processing unit 50. The filter processing executed by the filter processing unit 52 is the same as the filter processing executed by the filter processing unit 52 of the ultrasound diagnostic apparatus 100.

The image processing unit 50 generates the Doppler image data acquired at the different positions with the intervals of the step widths based on the filter-processed B-mode image data and the blood flow image data. The image processing unit 50 generates three-dimensional ultrasound image data composed of the Doppler image data of a plurality of frames acquired at different positions in the subject 54.

In this way, the filter processing based on the unnecessary component suppression filter characteristics is performed on the B-mode image data that is a source for generating the Doppler image data, whereby the stripe-like artifact appearing in the image displayed on the display unit 18 based on the three-dimensional ultrasound image data is suppressed.

### Configurations of Present Disclosure

### Configuration 1:

A three-dimensional ultrasound image processing apparatus comprising: an information processing unit that executes processing of acquiring spatial frequency distribution data in a depth direction for one frame in two-dimensional ultrasound image data of a plurality of frames constituting three-dimensional ultrasound image data, and processing of performing filter processing, which is based on filter characteristics determined in accordance with the spatial frequency distribution data and characteristics of transmitted ultrasound in a case in which each two-dimensional ultrasound image data is acquired, on each two-dimensional ultrasound image data.

### Configuration 2:

The three-dimensional ultrasound image processing apparatus according to configuration 1, in which the information processing unit searches for a spatial frequency corresponding to a maximal value of a distribution indicated by the spatial frequency distribution data in a search range determined in accordance with a pulse width of the transmitted ultrasound, and obtains, as the filter characteristics, characteristics for suppressing a level of the distribution indicated by the spatial frequency distribution data in a spatial frequency band including the spatial frequency that is searched for.

### Configuration 3:

The three-dimensional ultrasound image processing apparatus according to configuration 2, in which the information processing unit changes the filter characteristics in a case in which the maximal value of the distribution indicated by the spatial frequency distribution data does not satisfy a predetermined condition for the spatial frequency distribution data acquired from the two-dimensional ultrasound image data on which the filter processing has been performed.

### Configuration 4:

The three-dimensional ultrasound image processing apparatus according to any one of configurations 1 to 3, in which the information processing unit generates the two-dimensional ultrasound image data in sequence with elapse of time, and performs the filter processing, which is the same as the filter processing on the two-dimensional ultrasound image data of one frame generated earlier, on the two-dimensional ultrasound image data of one frame generated later.

### Configuration 5:

The three-dimensional ultrasound image processing apparatus according to configuration 4, in which the information processing unit performs the filter processing in which the filter characteristics are changed on the two-dimensional ultrasound image data of one frame generated much later in a case in which a condition related to an artifact is not satisfied for the two-dimensional ultrasound image data of the one frame generated later.

### Configuration 6:

The three-dimensional ultrasound image processing apparatus according to any one of configurations 1 to 5, in which the two-dimensional ultrasound image data is obtained by transmitting and receiving the ultrasound while performing scanning with a transmission beam of the ultrasound at a predetermined scanning interval, and the information processing unit performs the filter processing on the two-dimensional ultrasound image data in a case in which the scanning interval exceeds a predetermined scanning interval threshold value.

### Configuration 7:

The three-dimensional ultrasound image processing apparatus according to configuration 6, in which the scanning interval threshold value is determined based on the characteristics of the transmitted ultrasound.

### Configuration 8:

The three-dimensional ultrasound image processing apparatus according to any one of configurations 1 to 7, in which the filter characteristics are characteristics for making an attenuation amount in a spatial frequency band corresponding to a predetermined structure be equal to or less than an attenuation amount limit value.

### Configuration 9:

The three-dimensional ultrasound image processing apparatus according to any one of configurations 1 to 8, in which the two-dimensional ultrasound image data is data indicating a B-mode image on which a blood flow image is superimposed.

What is claimed is:

1. A three-dimensional ultrasound image processing apparatus comprising:
an information processing unit that executes
processing of acquiring spatial frequency distribution data in a depth direction for one frame in two-dimensional ultrasound image data of a plurality of frames constituting three-dimensional ultrasound image data, and
processing of performing filter processing, which is based on filter characteristics determined in accordance with the spatial frequency distribution data and characteristics of transmitted ultrasound in a case in which each two-dimensional ultrasound image data is acquired, on each two-dimensional ultrasound image data.

2. The three-dimensional ultrasound image processing apparatus according to claim 1,
wherein the information processing unit
searches for a spatial frequency corresponding to a maximal value of a distribution indicated by the spatial frequency distribution data in a search range determined in accordance with a pulse width of the transmitted ultrasound, and obtains, as the filter characteristics, characteristics for suppressing a level of the distribution indicated by the spatial frequency distribution data in a spatial frequency band including the spatial frequency that is searched for.

3. The three-dimensional ultrasound image processing apparatus according to claim 2,
wherein the information processing unit
changes the filter characteristics in a case in which the maximal value of the distribution indicated by the spatial frequency distribution data does not satisfy a predetermined condition for the spatial frequency distribution data acquired from the two-dimensional ultrasound image data on which the filter processing has been performed.

4. The three-dimensional ultrasound image processing apparatus according to claim 1,
wherein the information processing unit
generates the two-dimensional ultrasound image data in sequence with elapse of time, and
performs the filter processing, which is the same as the filter processing on the two-dimensional ultrasound image data of one frame generated earlier, on the two-dimensional ultrasound image data of one frame generated later.

5. The three-dimensional ultrasound image processing apparatus according to claim 4,
wherein the information processing unit
performs the filter processing in which the filter characteristics are changed on the two-dimensional ultrasound image data of one frame generated much later in a case in which a condition related to an artifact is not satisfied for the two-dimensional ultrasound image data of the one frame generated later.

6. The three-dimensional ultrasound image processing apparatus according to claim 1,
wherein the two-dimensional ultrasound image data is obtained by transmitting and receiving the ultrasound while performing scanning with a transmission beam of the ultrasound at a predetermined scanning interval, and
the information processing unit performs the filter processing on the two-dimensional ultrasound image data in a case in which the scanning interval exceeds a predetermined scanning interval threshold value.

7. The three-dimensional ultrasound image processing apparatus according to claim 6,
wherein the scanning interval threshold value is determined based on the characteristics of the transmitted ultrasound.

8. The three-dimensional ultrasound image processing apparatus according to claim 1,
wherein the filter characteristics are characteristics for making an attenuation amount in a spatial frequency band corresponding to a predetermined structure be equal to or less than an attenuation amount limit value.

9. The three-dimensional ultrasound image processing apparatus according to claim 1,
wherein the two-dimensional ultrasound image data is data indicating a B-mode image on which a blood flow image is superimposed.
